(19) United States
(12) Patent Application Publication
(10) Pub. No.: US 2025/0242334 A1
(43) Pub. Date: Jul. 31, 2025

(54) DEPOLYMERIZATION CATALYST AND PROCESS

(71) Applicant: Basell Poliolefine Italia S.r.l., Milano (IT)

(72) Inventors: Sandor Nagy, Seabrook, TX (US); Noel C. Hallinan, Loveland, OH (US)

(73) Assignee: Basell Poliolefine Italia S.r.l., Milano (IT)

(21) Appl. No.: 19/040,327

(22) Filed: Jan. 29, 2025

Related U.S. Application Data
(60) Provisional application No. 63/626,630, filed on Jan. 30, 2024.

(51) Int. Cl. B01J 23/30; B01J 21/04; B01J 21/16; B01J 23/28; B01J 37/02; B01J 37/04; B01J 37/30; C07C 4/22; C10B 53/07; C10B 57/06; C10B 57/14

(52) U.S. Cl. CPC B01J 23/30; B01J 21/04; B01J 21/16; B01J 23/28; B01J 37/0215; B01J 37/04; B01J 37/30; C07C 4/22; C10B 53/07; C10B 57/06; C10B 57/14; C07C 2523/28

(57) ABSTRACT

A supported catalyst for depolymerizing polymers and methods for making and using such catalyst. The supported catalyst comprises tungsten species supported on an alumina support, a molybdenum species supported on a smectite clay support, or a combination thereof. The supported catalyst is prepared by dissolving a tungstate salt or a molybdate salt in a solvent to form a first solution comprising cations and metal oxoanions, adding a Brønsted-Lowry acid to the first solution to form a second solution comprising a metal oxo acid, and contacting a support material with the second solution, wherein the support material is an alumina or a smectite clay, respectively. A mixture of a polyolefin-based feed stream and the supported catalyst can be added to a pyrolysis reaction zone under depolymerization conditions in the absence of oxygen to form a first vapor stream and first liquid stream comprising one or more olefin monomers.

### CROSS-REFERENCE TO RELATED APPLICATIONS

The application claims the benefit of priority to U.S. Provisional Patent Application No. 63/626,630 filed on Jan. 30, 2024, which is incorporated herein by reference in its entirety.

### FIELD OF THE INVENTION

This disclosure relates to methods of depolymerizing polyolefin-based material using a catalyst system comprising supported tungsten and/or molybdenum species.

### BACKGROUND OF THE INVENTION

Heightened standards of living and increased urbanization have led to an increased demand for polymer products, particularly polyolefin plastics. Polyolefins have been frequently used in commercial plastics applications because of their outstanding performance and cost characteristics. Polyethylene (PE), for example, has become one of the most widely used and recognized polyolefins because it is strong, extremely tough, and very durable. This allows for it to be highly engineered for a variety of applications. Similarly, polypropylene (PP) is mechanically rugged yet flexible, is heat resistant, and is resistant to many chemical solvents like bases and acids. Thus, it is ideal for various end-use industries, mainly for packaging and labeling, textiles, plastic parts, and reusable containers of various types.

The downside to the demand for polyolefin plastics is the increase in waste. Post-consumer plastic waste typically ends up in landfills, with about 12% being incinerated and about 9% being diverted to recycling. In landfills, most plastics do not degrade quickly, becoming a major source of waste that overburdens the landfill. Incineration is also not an ideal solution to treating the plastic wastes as incineration leads to the formation of carbon dioxide and other greenhouse gas emissions. As such, there has been much interest in developing methods of recycling plastic waste to reduce the burden on landfills while being environmentally friendly.

Recent advances have focused on converting plastic waste to useable products like fuel sources or commercially important raw materials. Methods of performing pyrolysis of the plastic waste stream followed by catalytic depolymerization have been developed to generate various products: gases, gasoline fractions, kerosene fractions, diesel fractions, and waxes. Thermal or pyrolytic depolymerization of plastic waste back to constituent oils for the purposes of recycling is an energy intensive process requiring temperatures up to 600° C. Various solid catalysts have been demonstrated to lower the temperature required for pyrolysis. U.S. Pat. No. 11,767,408 discloses depolymerizing polyolefins using supported metal oxides. Pub. Appl. US 2021/0061972 discloses depolymerizing plastics using a fluorinated alumina catalyst. PCT Pub. No. WO 2022/122596 discloses depolymerizing polyolefin-based waste material into useful petrochemical products using a composite of at least one zeolite catalyst with one or more solid inorganic co-catalyst(s). WO 2023/088861 discloses depolymerizing plastic waste using a silica supported heteropolyacids catalyst.

Though catalysts allow pyrolysis at lower temperatures than uncatalyzed pyrolysis, these lower temperatures may still be in the range of from 300° C. to 500° C. Such temperatures are still high enough that catalyst poisons present in most plastic waste will quickly degrade catalytic activity, such that continuous addition of fresh catalyst must be made. This high usage coupled with typical requirement of wt % levels rather than ppm levels of catalyst can lead to much higher costs for synthetic catalysts when compared to catalysts based on naturally occurring materials that are mined on the millions-of-tons scale annually for other applications. By virtue of their ready availability on a bulk scale, these latter materials can be up to an order of magnitude lower in cost than manufactured materials. Therefore, in instances where catalyst activity is maintained through continued addition of fresh catalyst, process economics favor the use of an inexpensive catalyst based on a naturally occurring material, even though it may have a lower initial catalytic activity than a more expensive synthetic catalyst.

While catalysts have been developed that demonstrate utility in depolymerizing plastic waste to produce more valuable olefinic feedstocks, there remains a need for catalysts capable of conversion of polyolefin-rich waste feeds to useful petrochemical products in spite of variable composition and/or quality of such waste feeds. Ideally, such catalysts could be formulated with commonly used raw materials and familiar techniques to provide additional options for processing waste plastics.

### SUMMARY OF THE INVENTION

The present disclosure provides a supported catalyst, a method for preparing a supported catalyst, and a method for using the catalyst. The method for making the supported catalyst comprises dissolving a metal oxo salt in a solvent to form a first solution comprising cations and metal oxoanions. A Brønsted-Lowry acid is added to the first solution in an amount sufficient to react with at least a portion of the metal oxoanions to form a second solution comprising a metal oxo acid. A support material is contacted with the second solution, wherein the support material is reactive with the metal oxo acid to form the supported catalyst. In some embodiments, the metal oxo salt is a tungstate salt and the support material is an alumina. In some embodiments, the metal oxo salt is a molybdate salt and the support material is a smectite clay.

The present disclosure also provides a supported catalyst produce by the foregoing method, wherein the supported catalyst is useful for depolymerizing polymers. A catalyst disclosed herein comprises a tungstic acid supported on an alumina support, a molybdic acid salt supported on a smectite clay support, or a combination thereof.

The present disclosure further provides for the use of a supported catalyst disclosed herein in a depolymerization process. The process comprises adding a polyolefin-based feed stream and the supported catalyst to a pyrolysis reaction zone to form a mixture. The mixture is reacted under depolymerization conditions in the absence of oxygen to form a first vapor stream and first liquid stream comprising one or more olefin monomers.

The foregoing has outlined rather broadly the features and technical advantages of the present invention in order that the detailed description of the invention that follows may be better understood. Additional features and advantages of the invention will be described hereinafter, which form the subject matter of the claims of the invention. It should be appreciated by those skilled in the art that the conception and specific embodiments disclosed may be readily utilized as a basis for modifying or designing other catalyst compositions and/or processes for carrying out the same purposes of the present invention. It should also be realized by those skilled in the art that such equivalent constructions do not depart from the spirit and scope of the invention as set forth in the appended claims. The novel features which are believed to be characteristic of the invention, both as to its compositions and methods, together with further objects and advantages will be better understood from the following description.

### BRIEF DESCRIPTION OF THE FIGURES

The claimed subject matter may be understood by reference to the following description taken in conjunction with the accompanying drawings, in which like reference numerals identify like elements, and in which:

FIG. 1 is a simplified flow diagram of a depolymerization process according to embodiments of the disclosure;

FIG. 2 shows overlaid graphs catalyst pyrolysis performance versus tungstate content of catalyst for comparative and inventive catalysts according to an embodiment of the disclosure;

FIG. 3 shows overlaid FTIR traces comparing water and tungstate salt solution before and after addition of alumina according to an embodiment of the disclosure;

FIG. 4 shows cation/anion content of tungstate salt solution at various levels of alumina additions according to an embodiment of the disclosure;

FIG. 5 shows the relationship between density and molarity of tungstate salt solution according to an embodiment of the disclosure;

FIG. 6 shows overlaid FTIR traces comparing various amounts of acid in tungstate salt solution in the ammonium ion spectral range according to an embodiment of the disclosure;

FIG. 7 shows overlaid FTIR traces comparing various amounts of acid in tungstate salt solution in the tungstate ion spectral range according to an embodiment of the disclosure;

FIG. 8 shows acidification of metatungstate solution with strong acid according to an embodiment of the disclosure;

FIG. 9A shows overlaid FTIR traces comparing water and meta tungstate solution before and after addition of alumina according to an embodiment of the disclosure;

FIG. 9B shows overlaid FTIR traces comparing water and meta tungstate solution before and after addition of strong acid according to an embodiment of the disclosure;

FIG. 9C shows overlaid FTIR traces comparing water and acidified meta tungstate solution before and after addition of alumina according to an embodiment of the disclosure;

FIG. 10A shows overlaid FTIR traces comparing water and tungstate solution before and after addition of alumina according to an embodiment of the disclosure;

FIG. 10B shows overlaid FTIR traces comparing water and tungstate solution before and after addition of strong acid according to an embodiment of the disclosure;

FIG. 10C shows overlaid FTIR traces comparing water and acidified tungstate solution before and after addition of alumina according to an embodiment of the disclosure;

FIG. 11A shows overlaid FTIR traces comparing water and hepta molybdate solution before and after addition of alumina according to an embodiment of the disclosure;

FIG. 11B shows overlaid FTIR traces comparing water and hepta molybdate solution before and after addition of strong acid according to an embodiment of the disclosure;

FIG. 11C shows overlaid FTIR traces comparing water and acidified hepta molybdate solution before and after addition of alumina according to an embodiment of the disclosure;

FIG. 12A shows overlaid FTIR traces comparing water and molybdate solution before and after addition of alumina according to an embodiment of the disclosure;

FIG. 12B shows overlaid FTIR traces comparing water and molybdate solution before and after addition of strong acid according to an embodiment of the disclosure;

FIG. 12C shows overlaid FTIR traces comparing water and acidified molybdate solution before and after addition of alumina according to an embodiment of the disclosure;

FIG. 13A shows overlaid FTIR traces comparing water and hepta molybdate solution before and after addition of bentonite according to an embodiment of the disclosure;

FIG. 13B shows overlaid FTIR traces comparing water and hepta molybdate solution before and after addition of strong acid according to an embodiment of the disclosure; and

FIG. 14 shows overlaid FTIR traces comparing water and hepta molybdate solution before and after addition of strong acid according to an embodiment of the disclosure.

While the disclosed process and composition are susceptible to various modifications and alternative forms, the drawings illustrate specific embodiments herein described in detail by way of example. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION OF THE INVENTION

Illustrative embodiments of the subject matter claimed below will now be disclosed. In the interest of clarity, some features of some actual implementations may not be described in this specification. It will be appreciated that in the development of any such actual embodiments, numerous implementation-specific decisions must be made to achieve the developer's specific goals, such as compliance with system-related and business-related constraints, which will vary from one implementation to another. Moreover, it will be appreciated that such a development effort, even if complex and time-consuming, would be a routine undertaking for those of ordinary skill in the art having the benefit of this disclosure.

The words and phrases used herein should be understood and interpreted to have a meaning consistent with the understanding of those words and phrases by those skilled in the relevant art. No special definition of a term or phrase, i.e., a definition that is different from the ordinary and customary meaning as understood by those skilled in the art, is intended to be implied by consistent usage of the term or phrase herein. To the extent that a term or phrase is intended to have a special meaning, i.e., a meaning other than the broadest meaning understood by skilled artisans, such a special or clarifying definition will be expressly set forth in the specification in a definitional manner that provides the special or clarifying definition for the term or phrase.

For example, the following discussion contains a non-exhaustive list of definitions of several specific terms used in this disclosure (other terms may be defined or clarified in a definitional manner elsewhere herein). These definitions are intended to clarify the meanings of the terms used herein. It is believed that the terms are used in a manner consistent with their ordinary meaning, but the definitions are nonetheless specified here for clarity.

### Definitions

As used herein, “a” or “an” when used in conjunction with the term “comprising” in the claims or the specification means one or more than one, unless the context dictates otherwise.

As used herein, “about” means the stated value plus or minus the margin of error of measurement or plus or minus 10% if no method of measurement is indicated.

As used herein, “acidified alumina” means alumina that has undergone a surface modification via acid treatment to enhance its surface acidity, in some instances improve its thermal stability and/or mechanical properties.

As used herein, “activated clay” describes clay or clay minerals, including, but not limited to, smectites or bentonites (which include montmorillonite, nontronite, beidellite and saponite); alumino-silicates, sepiolite, attapulgite (palygorskite); kaolins; and other fuller's earths which have been chemically treated with dilute acids (e.g., sulfuric acid) and thermally heat treated between 100° C. and 200° C.

As used herein, “char” refers to coke, a carbon-containing solid, that accumulates on the catalyst particles during pyrolysis.

As used herein, “comprise”, “have”, “include” and “contain” (and their variants) are open-ended linking verbs and allow the addition of other elements when used in a claim.

As used herein, “consisting essentially of” excludes additional material elements, but allows the inclusions of non-material elements that do not substantially change the nature of the invention.

As used herein, “consisting of” is closed and excludes all additional elements.

As used herein, “conversion” is used to denote the percentage of a component fed which disappears across a reactor.

As used herein, “feed stream” refers to a supply of polyolefin-based material for depolymerization. Depending on the depolymerization unit, the feed stream can be a continuous supply of material or a batch of material. The feed stream can be pure polyolefins or can be a mix of polyolefins with non-polyolefin components.

As used herein, “loading,” in the context of catalysts, refers to the deliberate deposition or impregnation of a specific amount of an active catalytic component onto a solid support material—e.g., the catalytic component is supported on or loaded onto a solid support. This is distinguished from merely being a mixture of the catalytic component and the solid support.

As used herein, “mol %” means the percentage of a single constituent as a percentage of all constituents of a mixture on a molar basis.

As used herein, “non-polyolefin components” refers to material present in a polyolefin-based feed, or waste, stream that can reduce the abilities of a zeolite to catalyze the depolymerization of the polyolefins that are present in the stream. Examples of non-polyolefin components include non-polyolefinic polymers with high oxygen and/or nitrogen content.

As used herein, “or” in the claims is used to mean “and/or” unless explicitly indicated to refer to alternatives only or if the alternatives are mutually exclusive.

As used herein, “post-consumer waste” refers to a type of waste produced by the end consumer of a material stream.

As used herein, “post-industrial waste” refers to a type of waste produced during the production process of a product.

As used herein, “reaction zone” refers to a chamber sufficiently enclosed to maintain selected operating conditions within the chamber to produce a desired reaction, such as a pyrolysis reaction zone or a condensing reaction zone. In some embodiments, each reaction zone can be a separate reactor. In some embodiments, a single vessel can contain a plurality of reaction zones.

As used herein, “reaction zone” refers to a chamber sufficiently enclosed to maintain selected operating conditions within the chamber to produce a desired reaction, such as depolymerization.

As used herein, “residence time” refers to the time needed to depolymerize a batch of polymer waste in a depolymerization unit.

As used herein, “strongly acidic” or “strong acid” refers to an acid that completely or nearly completely ionizes in water.

As used herein, “thermolysis” refers to a thermal depolymerization reaction occurring in the absence of oxygen.

As used herein, “waste stream” is a type of feed stream comprising material that has been discarded as no longer useful, including but not limited to, post-consumer and post-industrial waste.

As used herein, “zeolite” refers to an aluminosilicate mineral with a microporous structure. Zeolites are, in one aspect, useful as catalysts for the processes disclosed herein. Zeolites can occur naturally or can be produced industrially.

As used herein, the terms “depolymerization half time” or “half time of depolymerization” refer to the time needed to achieve a 50% loss of mass of a sample at a specific temperature during a TGA thermolysis reactions. The depolymerization half time is related to the residence time that would be needed for large scale industrial depolymerization reactors.

The embodiments illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps.

All concentrations herein are by weight percent (“wt %”) unless otherwise specified.

The following abbreviations are used herein:

| Abbreviation | Term |
|---|---|
| TGA | Thermogravimetric Gravimetric Analysis |
| WHSV | weight hourly space velocity |
| C₂⁼ | ethylene |
| C₃⁼ | propylene |
| mol % | Mole percentage |
| wt % | weight percentage |

### Method for Producing a Catalyst Composition

The present disclosure provides a method for making catalytic compositions for recycling polyolefin-based materials into commercially important raw materials. In some embodiments, a method for preparing a supported catalyst comprises dissolving a metal oxo salt in a solvent to form a first solution comprising cations and metal oxoanions. A Brønsted-Lowry acid is added to the first solution in an amount sufficient to react with at least a portion of the metal oxoanions to form a second solution comprising a metal oxo acid. A support material is contacted with the second solution, wherein the support material is reactive with the metal oxo acid to form the supported catalyst. In some embodiments, the metal oxo salt is a tungstate salt and the support material is an alumina, and the resulting supported catalyst is a tungstic acid catalyst. In some embodiments, the metal oxo salt is a molybdate salt and the support material is a smectite clay, and the resulting supported catalyst is a tungstic acid catalyst.

In some embodiments, the strong acid comprises an ion exchange resin such as, but not limited to, polystyrene-divinylbenzene sulfonated resin (e.g., Amberlyst™ 15, available from Sigma Aldrich, St. Louis, Missouri), gel type sulfonated polystyrene-divinylbenzene resin, macroporous sulfonated polystyrene-divinylbenzene resin, phenolic-based sulfonic acid resins, crosslinked polystyrene sulfonated resins with high acid capacity, surface sulfonated crosslinked polystyrene resins, acrylic matrix sulfonated resins, high crosslink density polystyrene-divinylbenzene sulfonated resins, or a combination thereof.

In some embodiments, the Brønsted-Lowry acid is a strong acid. In some embodiments, the Brønsted-Lowry acid is one or more members from the list consisting of hydrochloric acid (HCl), hydrobromic acid (HBr), hydroiodic acid (HI), nitric acid (HNO₃), perchloric acid (HClO₄), sulfuric acid (H₂SO₄), chloric acid (HClO₃), chlorosulfuric acid (HSO₃Cl), chlorosulfonic acid (HSO₃OH), fluorosulfuric acid (FSO₃H), oleum (fuming sulfuric acid), fluoroantimonic acid (HSbF₆), fluoroarsenic acid (HAsF₆), fluoroantimonous acid (HSbF₅), magic acid (HSO₃F/SbF₅), perfluorooctanesulfonic acid (PFOS), perfluorooctanoic acid (PFOA), triflic acid (CF₃SO₃H), methanesulfonic acid (CH₃SO₃H), ethanesulfonic acid (C₂H₅SO₃H), p-toluenesulfonic acid (C₆H₄CH₃SO₃H), camphorsulfonic acid (C₁₀H₁₆SO₄), and trifluoroacetic acid (CF₃COOH).

In some embodiments of the method for producing the metal oxo acid catalyst, the Brønsted-Lowry acid is mixed with the first solution under agitation. In some embodiments of the method for producing the metal oxo acid catalyst, the support material is contacted with the second solution under agitation. One skilled in the art would recognize that increased agitation improves contact efficiency in either a liquid/liquid mixture or a solid/liquid mixture. Such improved contact efficiency leads to decreased reaction and/or decreased energy consumption (e.g., reduced need to add heat). Such agitation can be implemented in laboratory or commercial scale operation via mechanical stirring (e.g., stirrers, impellers, or blades), magnetic stirring, a static mixer, ultrasound, a vortex inducer, sparging with inert gas, a recirculation loop, or any combination thereof.

In some embodiments of the method for producing the metal oxo acid catalyst, the support material is contacted with the second solution for a time period in the range of from 30 seconds to 1 hour, from 1 minutes to 30 minutes, or from 3 minutes to 10 minutes.

In some embodiments of the method for producing the metal oxo acid catalyst, the method may be performed at any temperature. In some embodiments of the method for producing the metal oxo acid catalyst, the method is performed at a temperature in the range of from 1° C. to 99° C., from 10° C. to 50° C., or from 20° C. to 25° C.

In some embodiments of the method for producing the metal oxo acid catalyst, the method may be performed at any pressure. In some embodiments of the method for producing the metal oxo acid catalyst, the method is performed at a pressure in the range of from 1 bar-a to 7 bar-g.

In some embodiments of the method for producing the metal oxo acid catalyst, the solvent is water.

### Tungstic Acid Catalyst

In some embodiments of the method for producing tungstic acid catalyst, the tungstate salt comprises one a more cations selected from the group consisting of: sodium, potassium, calcium, ammonium, lead, copper(II), iron(II), manganese(II), zinc, cadmium, silver, and cobalt. Tungstate salts can be selected from one or more of sodium tungstate (Na₂WO₄), potassium tungstate (K₂WO₄), calcium tungstate (CaWO₄), lead tungstate (PbWO₄), ammonium tungstate ((NH₄)₂WO₄), ammonium metatungstate ((NH₄₀)₆H₂W₁₂O₄₀), sodium metatungstate (Na₆H₂W₁₂O₄₀), sodium tungstate (Na₂WO₄), potassium tungstate (K₂WO₄), calcium tungstate (CaWO₄), ammonium tungstate ((NH₄)₂WO₄), lead tungstate (PbWO₄), copper(II) tungstate (CuWO₄), iron(II) tungstate (FeWO₄), manganese(II) tungstate (MnWO₄), zinc tungstate (ZnWO₄), cadmium tungstate (CdWO₄), silver tungstate (Ag₂WO₄), and cobalt tungstate (CoWO₄). In some embodiments, the tungstate salt comprises sodium or ammonium.

In some embodiments of the method for producing tungstic acid catalyst, the alumina support comprises gamma alumina, eta alumina, theta alumina, alpha alumina, or a combination thereof. In some embodiments, the alumina support is acidified. In some embodiments, the alumina support comprises acidified gamma alumina.

In some embodiments, the Brønsted-Lowry acid is a strong acid in the form of an ion exchange resin, having an exchange capacity, either stated in commercial specifications or measured in a laboratory. A mass of ion exchange resin in water would then be calculated to produce an aqueous solution having a proton concentration in mmols H+/g (“PC”), corresponding to the mass of mass of ion exchange resin. A mass of tungstate salt to be acidified would have a number of moles of metal or ammonium cationic groups (CG+) to be exchanged in mmols CG+/g (“CG”), corresponding to the mass of tungstate salt. The second solution contains sufficient ion exchange resin such that the ratio PC/CG is in the range of from 0.5-5, from 0.7-3 or from 0.9-2.

In some embodiments, the Brønsted-Lowry acid is a strong acid in liquid form, and the second solution comprises the acid in an amount in the range of from 0.01 M to 0.20 M, from 0.02 M to 0.16 M, from 0.03 M to 0.12 M, or from 0.04 M to 0.08 M, wherein M is molarity.

### Molybdic Acid Catalyst

In some embodiments of the method for producing molybdic acid catalyst, the molybdate salt comprises one a more cations selected from the group consisting of: sodium, potassium, calcium, ammonium, lead, copper(II), iron(II), manganese(II), zinc, cadmium, silver, and cobalt. Molybdate salts can be selected from one or more of sodium molybdate (Na₂MoO₄), potassium molybdate (K₂MoO₄), calcium molybdate (CaMoO₄), ammonium molybdate ((NH₄)₂MoO₄), lead molybdate (PbMoO₄), copper(II) molybdate (CuMoO₄), iron(II) molybdate (FeMoO₄), manganese(II) molybdate (MnMoO₄), zinc molybdate (ZnMoO₄), cadmium molybdate (CdMoO₄), silver molybdate (Ag₂MoO₄), cobalt molybdate (CoMoO₄), sodium heptamolybdate (Na₆Mo₇O₂₄), potassium heptamolybdate (K₆Mo₇O₂₄), ammonium heptamolybdate ((NH₄)₆Mo₇O₂₄), and calcium heptamolybdate (Ca₃Mo₇O₂₄·₈H₂O).

In some embodiments of the method for producing molybdic acid catalyst, the alumina support comprises gamma alumina, eta alumina, theta alumina, alpha alumina, or a combination thereof. In some embodiments, the alumina support is acidified. In some embodiments, the alumina support comprises acidified gamma alumina.

In some embodiments, the Brønsted-Lowry acid is a strong acid in the form of an ion exchange resin, having an exchange capacity, either stated in commercial specifications or measured in a laboratory. A mass of ion exchange resin in water would then be calculated to produce an aqueous solution having a proton concentration in mmols H+/g (“PC”), corresponding to the mass of mass of ion exchange resin. A mass of molybdate salt to be acidified would have a number of moles of metal or ammonium cationic groups (CG+) to be exchanged in mmols CG+/g (“CG”), corresponding to the mass of molybdate salt. The second solution contains sufficient ion exchange resin such that the ratio PC/CG is in the range of from 0.5-5, from 0.7-3 or from 0.9-2.

In some embodiments, the Brønsted-Lowry acid is a strong acid in liquid form, and the second solution comprises the acid in an amount in the range of from 0.01 M to 0.20 M, from 0.02 M to 0.16 M, from 0.03 M to 0.12 M, or from 0.04 M to 0.08 M, wherein M is molarity.

### Catalyst Composition

The present disclosure provides catalytic compositions for recycling polyolefin-based materials into commercially important raw materials. In some embodiments, a catalyst composition for depolymerizing a polyolefin-based feed stream in a depolymerization unit comprises or consists essentially of a tungstic acid supported on an alumina support, a molybdic acid salt supported on a smectite clay support, or a combination thereof. Supported on a support herein means that a specific amount of the relevant catalytically active acid is deliberately loaded or impregnated on the support as opposed to the catalytically active component just being mixed with the solid support material. The components of the supported catalyst work synergistically to increase the rate of depolymerization, thus reducing the amount of time the polyolefin-based feed stream spends in the depolymerization unit and/or the amount of energy consumed in to produce an equivalent product from an equivalent polymer recyclate feed stream.

### Tungstic Acid Catalyst

In some embodiments, a supported catalyst herein comprises a tungstic acid supported on an alumina support.

In some embodiments, the tungstic acid is derived from a tungstate salt that has been at least 50% protonated, at least 60% protonated, at least 70% protonated, at least 80% protonated, at least 90% protonated, or fully protonated.

In some embodiments, the tungstic acid is an isopoly acid comprising 1 to 20 metal atoms. An isopoly acid of tungsten consists only of oxoanions of a tungsten.

In some embodiments, the supported catalyst demonstrates a first pyrolysis rate (PR1) under a set of depolymerization conditions. A comparative catalyst comprising a tungstate salt corresponding to and in place of the tungstic acid demonstrates a second pyrolysis rate (PR2) under the same set of depolymerization conditions. In some embodiments, PR1/PR2 is greater than or equal to 1.5, greater than or equal to 2.0, greater than or equal to 2.5, or greater than or equal to 3.0.

In some embodiments of the tungstic acid catalyst, the alumina support comprises gamma alumina, eta alumina, theta alumina, alpha alumina, or a combination thereof. In some embodiments, the alumina support is acidified as is known to those skilled in the art. In some embodiments, the alumina support comprises acidified gamma alumina.

In some embodiments of the tungstic acid catalyst, the tungstic acid is supported on an alumina support in an amount greater than 8 wt %, or in the range of from 9 wt % to 20 wt %, from 10 wt % to 19 wt %, from 11 wt % to 18 wt %, from 12 wt % to 17 wt %, or from 13 wt % to 16 wt %, wherein weight percent is based on the total weight of the supported catalyst.

### Molybdic Acid Catalyst

In some embodiments, a supported catalyst herein comprises a molybdic acid supported on an alumina support.

In some embodiments, the molybdic acid is derived from a molybdate salt that has been at least 50% protonated, at least 60% protonated, at least 70% protonated, at least 80% protonated, at least 90% protonated, or fully protonated.

In some embodiments, the molybdic acid is an isopoly acid comprising 1 to 20 metal atoms. An isopoly acid of molybdenum consists only of oxoanions of a molybdenum.

In some embodiments, the supported catalyst demonstrates a first pyrolysis rate (PR1) under a set of depolymerization conditions. A comparative catalyst comprising a molybdate salt corresponding to and in place of the molybdic acid demonstrates a second pyrolysis rate (PR2) under the same set of depolymerization conditions. In some embodiments, PR1/PR2 is greater than or equal to 1.5, greater than or equal to 2.0, greater than or equal to 2.5, or greater than or equal to 3.0.

In some embodiments of the molybdic acid catalyst, the smectite clay support comprises bentonite, montmorillonite, beidellite, nontronite, saponite, hectorite, sauconite, swinefordite, or a combination thereof. In some embodiments the smectite clay is an activated smectite clay. In some embodiments, the clay support is acidified as is known to those skilled in the art. In some embodiments, the clay support comprises acidified bentonite.

In some embodiments of the molybdic acid catalyst, the molybdic acid is supported on an smectite clay support in an amount greater than 8 wt %, or in the range of from 9 wt % to 20 wt %, from 10 wt % to 19 wt %, from 11 wt % to 18 wt %, from 12 wt % to 17 wt %, or from 13 wt % to 16 wt %, wherein weight percent is based on the total weight of the supported catalyst.

### Process Using the Catalyst Composition

In some embodiments of the present disclosure, the supported catalyst is a tungstic acid catalyst, a molybdic acid catalyst, or a combination thereof, as disclosed herein. The presently described supported catalysts can be used to thermally degrade or depolymerize a feed stream comprising material with a single polyolefin component or a mixture of polyolefin components in any amount. Any polyolefin can be present in the feed stream, including but not limited to, polyethylene (both high and low density), polypropylene, ethylene-propylene copolymers, polybutene-1, polyisobutene, and copolymers thereof. Further, the feed stream is not limited to any particular form so films, foams, textiles or other shaped material can be treated with the described methods. The polyolefins can be obtained from waste streams, including post-consumer waste streams, post-industrial waste streams, or combinations thereof.

In some embodiments, a process for depolymerizing polymers comprises adding a polyolefin-based feed stream and the supported catalyst comprising a tungstic acid catalyst, a molybdic acid catalyst, or a combination thereof, as disclosed herein, to a pyrolysis reaction zone to form a mixture. The mixture is reacted under depolymerization conditions in the absence of oxygen to form a first vapor stream and first liquid stream comprising char. The first vapor stream is added to a first condensation zone, wherein the first vapor product is subjected to condensing conditions to form a second vapor product and a second liquid product comprising one or more olefin monomers.

In further embodiments of the process for depolymerizing polymers, the polyolefin feed stream comprises polyethylene, polypropylene, or a combination thereof. In some embodiments, the polyolefin feed stream comprises up to 20 wt %, up to 15 wt %, up to 10 wt %, or up to 5 wt % of an impurity, wherein the weight percentage is based on the total weight of the polyolefin feed stream. In some embodiments, the impurity comprises one or more members of the group consisting of polyethylene terephthalate, polystyrene, water, chlorine, or a combination thereof.

In some embodiments of the process for depolymerizing polymers, the depolymerization conditions comprise a temperature in the range of from 250° C. to 600° C., from 400° C. to 600° C., from 425° C. to 550° C., or from 450° C. to 500° C., a pressure in the range of from 100 kPa to 1,000 kPa, from 100 kPa to 700 kPa, from 150 kPa) to 600 kPa, or from 200 kPa to 500 kPa, or a combination thereof.

In some embodiments of the process for depolymerizing polymers, the condensing conditions independently comprise a temperature in the range of from 20° C. to 100° C., from 30° C. to 90° C., or from 40° C. to 80° C., a pressure in the range of from 30 kPa to 200 kPa, from 50 kPa to 170 kPa, or from 70 kPa to 130 kPa, or a combination thereof.

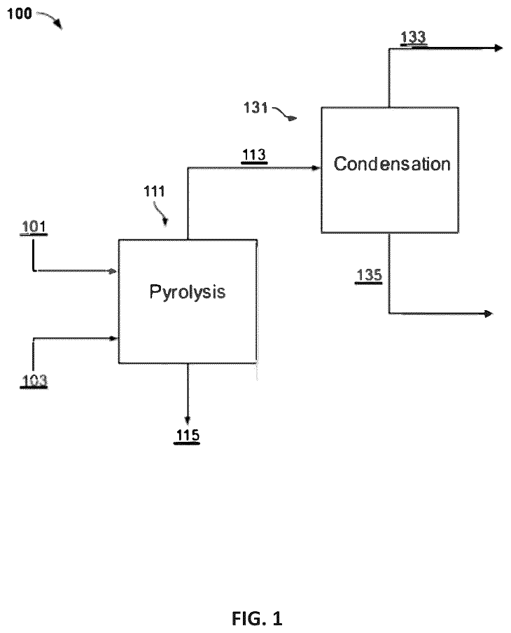

FIG. 1 shows an embodiment of a depolymerization process 100 comprising a pyrolysis reactor 111 and a condensation unit 131. Such embodiment would also include any common equipment associated with distillation columns, including, but not limited to, heat exchangers, pumps, valves, reflux loops, and the like, which are omitted for simplicity.

Polymer recyclate feed 101, a catalyst composition disclosed herein 103, and an inert gas 105, such as nitrogen, are fed to pyrolysis reactor 111. The mixture of catalyst and polymer recyclate are subjected to depolymerization conditions in a reaction zone within pyrolysis reactor 111 to produce a first vapor stream 113 and a liquid stream 115 comprising char.

The vapor stream 113 is fed to condensation unit 131 to be cooled to form a second vapor stream 133 and a second liquid product stream 135 comprising one or more olefin monomers.

### Certain Embodiments

Disclosed herein is a method for preparing a supported catalyst useful for depolymerization processes. The method comprises dissolving a metal oxo salt in a solvent to form a first solution comprising cations and metal oxoanions. A Brønsted-Lowry acid is added to the first solution in an amount sufficient to react with at least a portion of the metal oxoanions to form a second solution comprising a metal oxo acid. A support material is contacted with the second solution, wherein the support material is reactive with the metal oxo acid to form the supported catalyst. In some embodiments, the metal oxo salt is a tungstate salt and the support material is an alumina. In some embodiments, the metal oxo salt is a molybdate salt and the support material is a smectite clay. In some embodiments, the catalyst is a mixture of the reaction product of an acidified tungstate salt loaded onto an alumina support and the reaction product of an acidified molybdate salt loaded onto a smectite clay support.

In some embodiments, the method for preparing a supported catalyst is further described by one or more of the following:
a) the tungstate salt and the molybdate salt each independently comprises one a more cations selected from the group consisting of: sodium, potassium, calcium, ammonium, lead, copper(II), iron(II), manganese(II), zinc, cadmium, silver, and cobalt;
b) the tungstate salt and the molybdate salt each independently comprises sodium and/or ammonium;
c) the Brønsted-Lowry acid is a strong acid;
d) the Brønsted-Lowry acid is one or more members from the list consisting of hydrochloric acid (HCl), hydrobromic acid (HBr), hydroiodic acid (HI), nitric acid (HNO₃), perchloric acid (HClO₄), sulfuric acid (H₂SO₄), chloric acid (HClO₃), chlorosulfuric acid (HSO₃Cl), chlorosulfonic acid (HSO₃OH), fluorosulfuric acid (FSO₃H), oleum (fuming sulfuric acid), fluoroantimonic acid (HSbF₆), fluoroarsenic acid (HAsF₆), fluoroantimonous acid (HSbF₅), magic acid (HSO₃F/SbF₅), perfluorooctanesulfonic acid (PFOS), perfluorooctanoic acid (PFOA), triflic acid (CF₃SO₃H), methanesulfonic acid (CH₃SO₃H), ethanesulfonic acid (C₂H₅SO₃H), p-toluenesulfonic acid (C₆H₄CH₃SO₃H), camphorsulfonic acid (C₁₀H₁₆SO₄), and trifluoroacetic acid (CF₃COOH);
e) the Brønsted-Lowry acid is a strong acid comprising an ion exchange resin such as, but not limited to, polystyrene-divinylbenzene sulfonated resin (e.g., Amberlyst™ 15, available from Sigma Aldrich, St. Louis, Missouri), gel type sulfonated polystyrene-divinylbenzene resin, macroporous sulfonated polystyrene-divinylbenzene resin, phenolic-based sulfonic acid resins, crosslinked polystyrene sulfonated resins with high acid capacity, surface sulfonated crosslinked polystyrene resins, acrylic matrix sulfonated resins, high crosslink density polystyrene-divinylbenzene sulfonated resins, or a combination thereof.
f) the alumina support comprises gamma alumina, eta alumina, theta alumina, alpha alumina, or a combination thereof, wherein in further embodiments, the alumina support is acidified, and in further embodiments, the support is an acidified gamma alumina;
g) the smectite clay support comprises bentonite, montmorillonite, beidellite, nontronite, saponite, hectorite, sauconite, swinefordite, or a combination thereof, wherein in further embodiments, the smectite clay support is acidified, and in further embodiments, the support is a bentonite or an acidified bentonite;
h) the first solution comprises the metal oxo salt in an amount in the range of from 0.01 M to 0.20 M, from 0.02 M to 0.16 M, from 0.03 M to 0.12 M, or from 0.04 M to 0.08;
i) the second solution comprises the Brønsted-Lowry acid in the form of an ion exchange resin, having an exchange capacity, either stated in commercial specifications or measured in a laboratory, wherein:
   1) a mass of ion exchange resin in water would then be calculated to produce an aqueous solution having a proton concentration in mmols H+/g (“PC”), corresponding to the mass of mass of ion exchange resin;
   2) a mass of tungstate salt or molybdate salt to be acidified would have a number of moles of metal or ammonium cationic groups (CG+) to be exchanged in mmols CG+/g (“CG”), corresponding to the mass of tungstate salt or molybdate salt, respectively; and
   3) the second solution contains sufficient ion exchange resin such that the ratio PC/CG is in the range of from 0.5-5, from 0.7-3 or from 0.9-2;
j) the second solution comprises the Brønsted-Lowry acid in an amount in the range of from 0.01 M to 0.20 M, from 0.02 M to 0.16 M, from 0.03 M to 0.12 M, or from 0.04 M to 0.08;
k) the Brønsted-Lowry acid is mixed with the first solution under agitation;
l) the metal oxo acid catalyst, the support material is contacted with the second solution under agitation;
m) the Brønsted-Lowry acid is mixed with the first solution for a time period in the range of from 30 seconds to 1 hour, from 1 minutes to 30 minutes, or from 3 minutes to 10 minutes;
n) the metal oxo acid catalyst, the support material is contacted for a time period in the range of from 30 seconds to 1 hour, from 1 minutes to 30 minutes, or from 3 minutes to 10 minutes;
o) the method is performed at a temperature in the range of from 1° C. to 99° C., from 10° C. to 50° C., or from 20° C. to 25° C.;
p) the method is performed at a pressure in the range of from 1 bar-a to 7 bar-g; and
q) the solvent is water.

In another aspect, a supported catalyst useful for depolymerization processes is provided. In some embodiments, a supported catalyst is the product of any one of the foregoing embodiments of a method to make a supported catalyst. Alternately, a supported catalyst described herein comprises a tungstic acid supported on an alumina support, a molybdic acid supported on a smectite clay support, or a combination thereof. In some embodiments, the supported catalyst is further described by one or more of the following:
a) the tungstic acid and/or the molybdic acid are at least 50% protonated, at least 60% protonated, at least 70% protonated, at least 80% protonated, at least 90% protonated, or fully protonated;
b) the tungstic acid and/or the molybdic acid are isopoly acid comprising 1 to 20 metal atoms;
c) the tungstic acid is fully protonated, and the supported catalyst demonstrates a first pyrolysis rate (PR1), a comparative catalyst comprising a tungstate salt corresponding to and in place of the tungstic acid demonstrates a second pyrolysis rate (PR2), and PR1/PR2 is greater than or equal to 1.5, greater than or equal to 2.0, greater than or equal to 2.5, or greater than or equal to 3.0;
d) the molybdic acid is fully protonated, and the supported catalyst demonstrates a first pyrolysis rate (PR1), a comparative catalyst comprising a molybdate salt corresponding to and in place of the molybdic acid demonstrates a second pyrolysis rate (PR2), and PR1/PR2 is greater than or equal to 1.5, greater than or equal to 2.0, greater than or equal to 2.5, or greater than or equal to 3.0.
e) the alumina support comprises gamma alumina, eta alumina, theta alumina, alpha alumina, or a combination thereof, wherein in further embodiments, the alumina support is acidified, and in further embodiments, the support is an acidified gamma alumina;
f) the smectite clay support comprises bentonite, montmorillonite, beidellite, nontronite, saponite, hectorite, sauconite, swinefordite, or a combination thereof, wherein in further embodiments, the smectite clay support is acidified, and in further embodiments, the support is a bentonite or an acidified bentonite;
g) the tungstic acid is supported on an alumina support in an amount greater than 8 wt %, or in the range of from 9 wt % to 20 wt %, from 10 wt % to 19 wt %, from 11 wt % to 18 wt %, from 12 wt % to 17 wt %, or from 13 wt % to 16 wt %, wherein weight percent is based on the total weight of the supported catalyst; and
h) the molybdic acid salt supported on a smectite clay support in an amount greater than 8 wt %, or in the range of from 9 wt % to 20 wt %, from 10 wt % to 19 wt %, from 11 wt % to 18 wt %, from 12 wt % to 17 wt %, or from 13 wt % to 16 wt %, wherein weight percent is based on the total weight of the supported catalyst.

In another aspect, a depolymerization process using the supported catalyst is provided. The process comprises adding a polyolefin-based feed stream and a supported catalyst according to any one of the above embodiments of a supported catalyst to a pyrolysis reaction zone to form a mixture. The mixture is reacted under depolymerization conditions in the absence of oxygen to form a first vapor stream and first liquid stream comprising char. The first vapor stream is added to a condensation zone wherein heat is removed to form a second vapor stream and a second liquid stream comprising one or more olefin monomers. In some embodiments, the process is further described by one or more of the following:
a) the polyolefin feed stream comprises polyethylene, polypropylene, or a combination thereof.
b) the depolymerization conditions comprise a temperature in the range of from 400° C. to 600° C., a pressure in the range of from 1.0 barg (100 kPa) to 7.0 barg (700 kPa), or a combination thereof.
c) the conditions in the condensing zone comprise a temperature in the range of from 20° C. to 100° C., from 30° C. to 90° C., or from 40° C. to 80° C., a pressure in the range of from 30 kPa to 200 kPa, from 50 kPa to 170 kPa, or from 70 kPa to 130 kPa, or a combination thereof; and
d) the catalyst is present in an amount in the range of from greater than 0 wt % to 20 w %, from 0.5 wt % to 10 w %, or from 1 wt % to 5 w %.

The presently disclosed supported catalysts and methods of making and using them are exemplified with respect to the examples below. These examples are included to demonstrate embodiments of the appended claims. However, these are exemplary only, and the invention can be broadly applied to embodiments not demonstrated in the examples. Those of skill in the art should appreciate that many changes can be made in the specific embodiments which are disclosed and still obtain a like or similar result without departing from the spirit and scope of the disclosure herein. In no way should the following examples be read to limit, or to define, the scope of the appended claims.

### EXAMPLES

The following examples are included to demonstrate embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Background

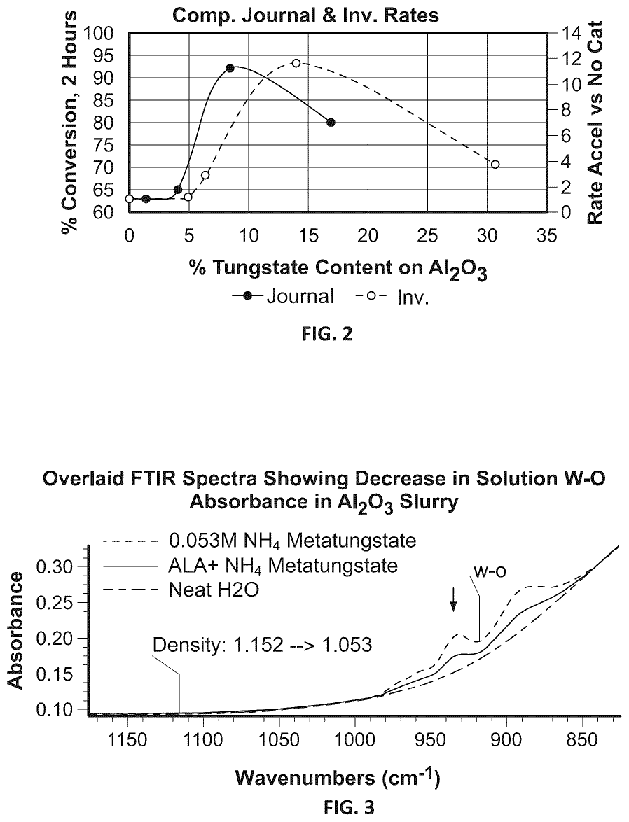

Pyrolysis of HDPE catalyzed by alumina (Al₂O₃) catalysts impregnated with tungstate is described in Park, Sung & Kang, Seung & Min, Hyung-Ki & Seo, Myung-gi & Kweon, Sungjoon & Park, Min & Choi, Young & Lee, Jae, (2022), “Catalytic pyrolysis of HDPE over WOₓ/Al₂O₃: Effect of tungsten content on the acidity and catalytic performance,” Molecular Catalysis, 528, 112439. 10.1016/j.mcat.2022.112439. Catalysts were prepared by incipient wetness technique from a starting ammonium metatungstate, (NH₄)₆H₂W₁₂O₄₀ solution. The catalysts had tungstate loadings as indicated by the data points in FIG. 2 (labeled as “Journal”). The pyrolysis rate, as measured by percent conversion over a 2 hour run period, was a function of tungstate loading on Al₂O₃, reaching a maximum at around 8 wt % and decreasing thereafter as shown in FIG. 2 (labeled as “Journal” and referencing the left vertical axis).

Catalyst preparations as described in the journal article were repeated with ammonium tungstate with the exception that a slurry-based method was used. Using this method, intermediate solution FTIR spectra of small aliquots of filtered slurry could be obtained. The decrease in absorbance of W—O FTIR bands can then be conveniently used to quantify the disappearance of tungstate from solution as it is adsorbed onto solid Al₂O₃ support as shown in FIG. 3. In addition, measurement of the density of tungstate solution before and after slurrying with Al₂O₃ provides a second method of quantification of tungstate loss from solution. Solution density can be easily measured gravimetrically with an accuracy of >+/−0.01 with a 250 μl gastight syringe.

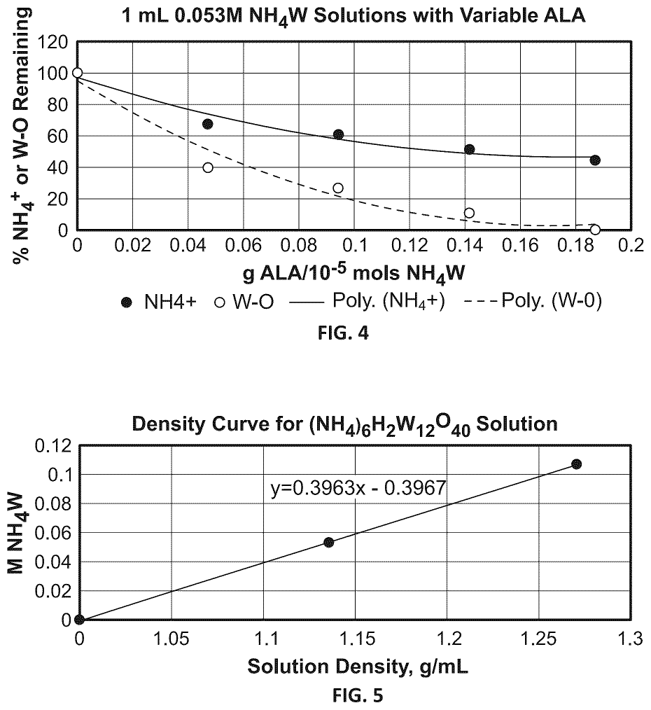

Using these quantification methods, the maximal loading of ammonium tungstate on acidic Al₂O₃ was about 14 wt %, based on the total weight of the tungstate and support. Attempts to achieve higher loading from slurries of very concentrated tungstate solution were unsuccessful. A profile of solution FTIR-determined tungstate loading is shown in FIG. 4 in which 14 wt % maximum tungstate loading corresponds to about 5.5 e⁻⁵ mols tungstate/g acidic Al₂O₃ (acidic Al₂O₃ indicated as ALA). The lower curve indicates the complete disappearance of tungstate ion, while the upper curve shows that some ammonium ion remains in solution, possibly as ammonium hydroxide, after all tungstate ion is impregnated on the support.

### FTIR Method

FTIR spectroscopy was carried out as follows. An iS50 FTIR spectrometer equipped with a DTGS detector from Thermo Scientific was used for FTIR spectral acquisition. The sample compartment was fitted with an attenuated total reflectance (ATR) accessory. The ATR cell was obtained from Pike Technologies and was equipped with a 3-bounce zinc selenide (ZnSe) crystal. A sample volume of about 0.05 mLs was required for analysis. Those familiar with FTIR spectroscopy will recognize that when multiple spectra are overlaid in the same plot, these spectra can be shown with similar baselines or offset baselines. This choice is purely for optimal viewing of changes occurring and does not impact peak height or peak absorbance measurements as such measurements are made from the baseline to the top of the peak.

### TGA Method

Unless otherwise noted, the depolymerization unit was a Thermogravimetric Gravimetric Analysis (TGA) instrument. For the TGA thermolysis reactions, the uniform samples were heated under nitrogen at 10K/min to a depolymerization temperature of 400° C. in a Mettler Toledo TGA/DSC 3+ (Mettler Toledo, Columbus, Ohio) and held for 1 hour. The depolymerization half time at a specific temperature, defined as the time needed to achieve a 50% loss of mass, was recorded directly if the value was less than 60 min, or determined under the assumption of first order decomposition kinetics as t_{1/2}=0.693/k, where k is the first order rate constant determined graphically using a Ln (Co/C) vs time plot.

The depolymerization half time is related to the residence time needed in a large scale depolymerization unit. The shorter the half time, the shorter the residence time for a batch of a polymer feed in a depolymerization unit, and the faster the depolymerization rate k.

As before, the various samples were processed using TGA as the depolymerization unit, and samples were prepared by melt-compounding the polymer with 2.5% catalyst loading in a HAAK MiniCTW compounder at 200° C. and 200 RPM for 5 minutes. The samples were heated under nitrogen at 10K/min to a depolymerization temperature of 400° C. and held for 1 hour.

### TGA Results

The prepared catalysts had tungstate loadings as indicated by the data points in FIG. 2 (labeled as “Prepared”). The pyrolysis rate, as measured by rate acceleration vs. no catalyst over a 2 hour run period, was a function of tungstate loading on Al₂O₃, reaching a maximum at around 14 wt % and decreasing thereafter as shown in FIG. 2 (labeled as “Prepared” and referencing the right vertical axis). Rate data from these experiments are overlaid in FIG. 2 with the journal article data and it can be observed that similar behavior takes place. The highest tungstate “loading” catalyst of around 30 wt % was prepared via incipient wetness as this loading was not accessible via slurry. Without wishing to be bound by any particular theory, it is believed that about 14 wt % tungstate was actually impregnated on the Al₂O₃ support while the remainder was simply mixed with the Al₂O₃ support. This surplus mixed tungstate appears to have an inhibitory effect on pyrolysis rate.

The journal does not disclose that a limitation on tungstate activity is a function of true impregnation through the incipient wetness preparation technique. The Journal data in FIG. 2 shows a “no catalyst” baseline of about 63% conversion and a maximum conversion of about 93% at a tungstate loading of about 8 wt %. The catalysts prepared by the slurry preparation technique herein demonstrate a rate acceleration of about 11.5 at a tungstate loading of about 14 wt %. Comparison of the current tungstate catalyst prepared by the slurry technique to the examples in the journal show a comparable performance profile of conversion rate versus metal salt content. It will be shown in the examples below that acidification of the metal salts can produce a significant improvement in conversion performance relative to untreated metal salts.

An unexpected discovery was that a simple pre-acidification step of the tungstate solution prior to slurrying with Al₂O₃ leads to a up to a 3-fold improvement in pyrolysis rate as compared to the unmodified tungstate. This acidification step can be carried out either heterogeneously or homogeneously. In the heterogeneous phase, Amberlyst™ 15 resin (available from Sigma Aldrich, St. Louis, Missouri) is shaken with a tungstate solution at room temperature for 1-5 minutes. The tungstate solution is then decanted or syringed off the resin and slurried with Al₂O₃. In the homogeneous phase, the tungstate solution can be mixed with a mineral acid solution (such as H₂SO₄) prior to impregnation onto Al₂O₃.

Another unexpected discovery was that the about 3-fold improvement in pyrolysis rate after a pre-acidification step applies not only to those catalysts in which tungstate is impregnated on Al₂O₃ but also molybdates impregnated on bentonite (F-20X, Engineered Clays Corp., Jackson, Mississippi). In contrast, molybdates, either unmodified or acidified, when impregnated on Al₂O₃, show no catalytic activity, and acidified tungstates actually show diminished rate acceleration relative unmodified tungstates when impregnated on bentonite. That is to say, that Group 6 metal oxo salts are not interchangeable in the methods or catalysts disclosed herein.

Yet another unexpected discovery was that acidified alumina (designated “ALA” herein) facilitated a higher tungstate loading than basic alumina (designated “ALB” herein). When ALB was used as support, the achievable tungstate loading via the slurry method was only about half the tungstate loading demonstrated for ALA. Similarly, pyrolysis rate acceleration with this ALB catalyst was only half that of the ALA catalyst. However, use of acidified tungstate solution with ALB support led to tungstate loadings that substantially matched the 14 wt % achieved with ALA as the support.

### Group VIA Salts Used

A list of tungstate and molybdate salts that were used are shown in Table 1, below. The metal oxo salts listed in Table 1 exemplary and not exhaustive, and other tungstate and molybdate salts are included in the methods and catalyst compositions disclosed herein. Other not listed are also contemplated as being part of this invention. Table 1 also shows the measured densities and calculated molarities for 25 wt % aqueous stock solutions prepared for each salt. Tungstic acid, though insoluble in H₂O, is included for later discussion purposes. A density curve for (NH₄)₆H₂W₁₂O₄₀ is shown in FIG. 5. Similar density curves for other solutions allows molarities in slurried solution to be calculated from density measurements.

TABLE 1

| | | MW | Salt in | Density | Molarity |
|---|---|---|---|---|---|
| Metal Oxo Salt | Formula | (g/mol) | Soln. (wt %) | (g/cc) | (M) |
| Ammonium metatungstate | (NH₄)₆H₂W₁₂O₄₀ | 2956 | 25 | 1.299 | 0.107 |
| Sodium metatungstate | Na₆W₁₂O₃₉ | 2968 | 25 | 1.276 | 0.107 |
| Sodium tungstate | Na₂WO₄ | 330 | 25 | 1.269 | 0.77 |
| Ammonium heptamolybdate | (NH₄)₆Mo₇O₂₄ | 1235 | 25 | 1.233 | 0.2 |
| Ammonium molybdate | (NH₄)₂MoO₄ | 196 | 25 | 1.19 | 1.21 |
| Sodium molybdate | Na₂MoO₄ | 242 | 25 | 1.284 | 1.17 |
| Tungstic acid | H₂WO₄ | 250 | 0 | NA | NA |

### Acidification of Tungstates and Molybdates

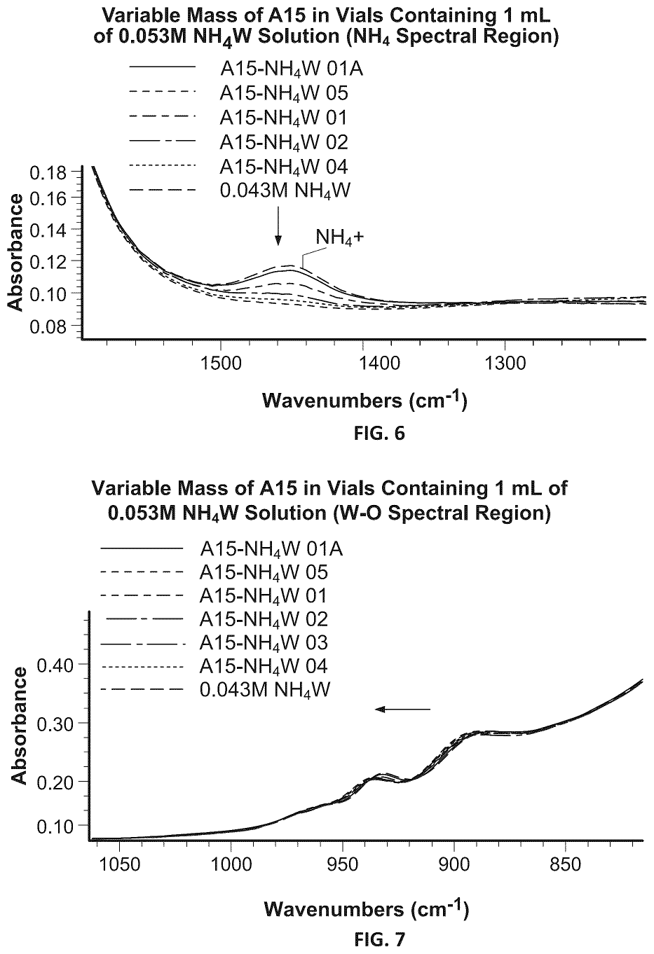

As (NH₄)₆H₂W₁₂O₄₀ in aqueous solution displays a discrete NH₄⁺ FTIR absorbance band at around 1450 cm⁻¹, disappearance of this band as ammonium ion exchanges out of solution with proton onto strong acid resin provides a convenient method of quantifying the extent of acidification. This is shown in the overlaid spectra in FIG. 6 associated with individual experiments in which variable mass Amberlyst™ 15 resin was slurried with similar volume tungstate solution in vials and sampled after 5 minutes.

These same spectra, now showing the spectral region containing W—O absorbances, are now presented in FIG. 7. While a slight peak shift to higher frequency was observed, there was no absorbance decrease that would be indicative of tungstate ion adsorption onto the acid resin.

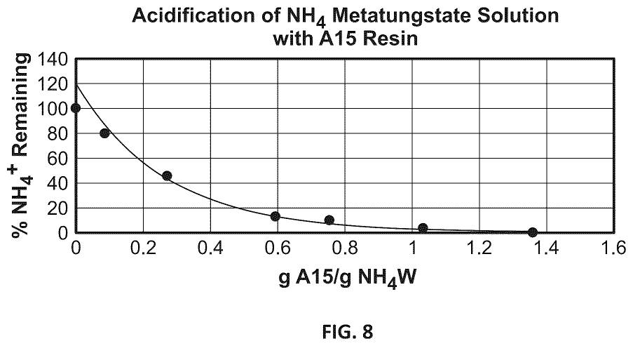

The spectra in FIG. 6 and FIG. 7 are associated with the data in FIG. 8 as shown in Table 2, below. The data in FIG. 8, obtained from peak height measurements of spectra as shown in FIG. 6, show an exponential dependence of ammonium ion exchange from solution with close to 100% exchange being achieved at a loading of about 1 g of dry Amberlyst™ 15 per gram of (NH₄)₆H₂W₁₂O₄₀.

TABLE 2

| Annotation in | Data in FIG. 8 |
|---|---|
| FIGS. 6 & 7 | (g A15/g NH₄W) |
| 0.043M NH₄W | 0 |
| A15-NH₄W 01A | 0.09 |
| A15-NH₄W 01 | 0.27 |
| A15-NH₄W 02 | 0.6 |
| A15-NH₄W 03 | 0.76 |
| A15-NH₄W 04 | 1.03 |
| A15-NH₄W 05 | 1.36 |

These rigorously quantified data, coupled with the complete disappearance of the NH₄⁺ peak from solution strongly suggest that complete acidification of (NH₄)₆H₂W₁₂O₄₀ is taking place per the equation below to give metatungstic acid. It should be noted that this acid is not commercially available.

(NH₄)₆H₂W₁₂O₄₀(soln)+A15(solid)→H₈W₁₂O₄₀(soln)+A15-NH₄⁺(solid)

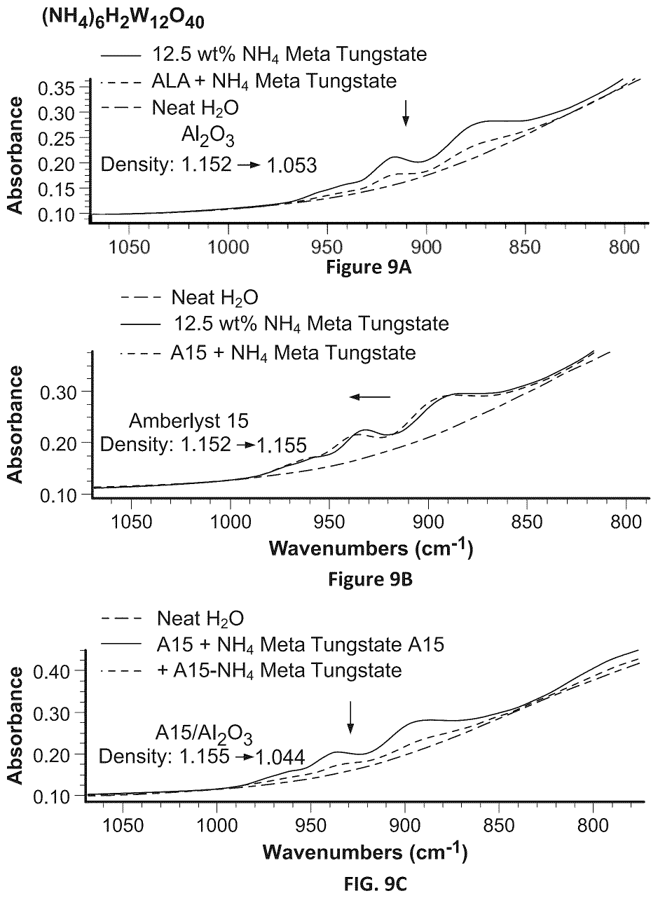

The spectroscopic behavior of (NH₄)₆H₂W₁₂O₄₀ solution with regard to its impregnation onto ALA is shown in FIG. 9A, its acidification with Amberlyst™ 15 is shown in FIG. 9B, and to acidified (NH₄)₆H₂W₁₂O₄₀ solution impregnation onto ALA is shown in FIG. 9C in which the W—O spectral region is displayed. In addition, before and after measured solution densities for each step are annotated on the spectra. The combined spectroscopic and density data show that both “as is” (NH₄)₆H₂W₁₂O₄₀ solution and acidified (NH₄)₆H₂W₁₂O₄₀ solution have similar loadings on ALA. The data also show that the acidification step leads to only ammonium ion exchange and no tungstate ion adsorption onto the resin. As data in a later section will show, the acidified (NH₄)₆H₂W₁₂O₄₀ on ALA has a 3-fold higher catalytic activity than the “as is” (NH₄)₆H₂W₁₂O₄₀ version. Similar acidifications of other tungstates and molybdates with Amberlyst™ 15 (A15) led to similar results with no adsorption onto the resin noted as shown in Table 3. All four materials and respective solutions were slurried in a ratio of 1 g A15 to 6 ml of solution.

TABLE 3

| | Strong Acid | Density (g/cc) | | Adsorption |
|---|---|---|---|---|
| Material | Resin | Before | After | (wt %) |
| (NH₄)₆H₂W₁₂O₄₀ | A15 | 1.152 | 1.155 | 0 |
| Na₂WO₄ | A15 | 1.125 | 1.118 | 0 |
| (NH₄)₂Mo₄ | A15 | 1.103 | 1.102 | 0 |
| (NH₄)₆Mo₇O₂₄ | A15 | 1.114 | 1.113 | 0 |

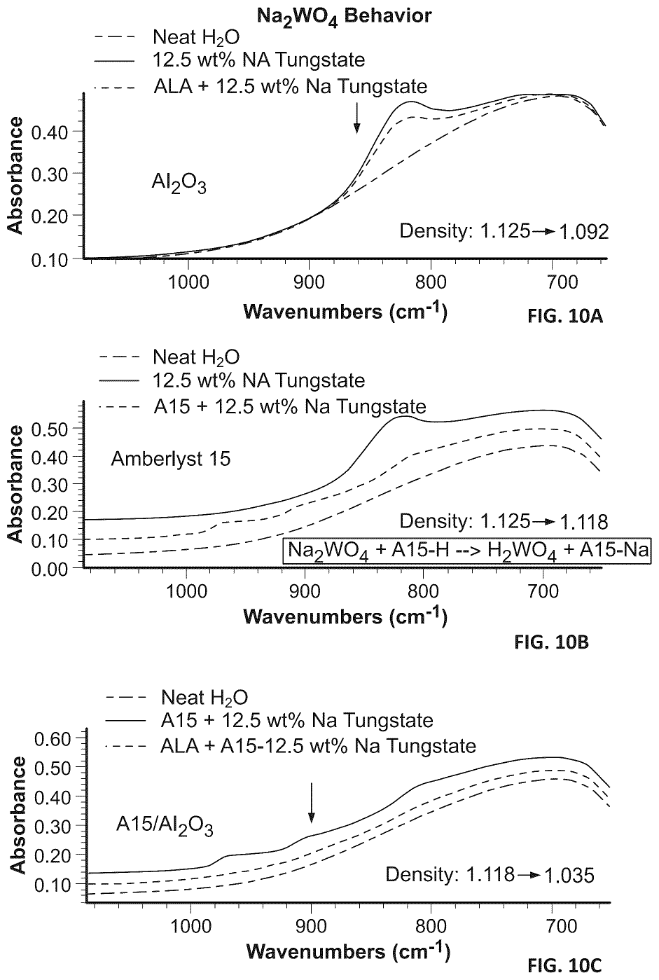

While as shown in FIG. 7, acidification of metatungstate leads to only minimal change in W—O FTIR absorbance, such is not the case for acidification of the simpler salt, Na₂WO₄. In this case, as noted in FIG. 10A-FIG. 10C, W—O bonding undergoes significant changes. Also noted is that while Na₂WO₄ displays relatively poor adsorption onto ALA based on modest solution density decrease as shown in FIG. 10A, the acidified version now shows adsorption that rivals that of the metatungstate as shown in FIG. 10C. This further points to the non-obviousness of behavior of acidified tungstates and molybdates and also shows that an apparent soluble version of tungstic acid (H₂WO₄) is generated in-situ, even though commercial solid H₂WO₄ is insoluble.

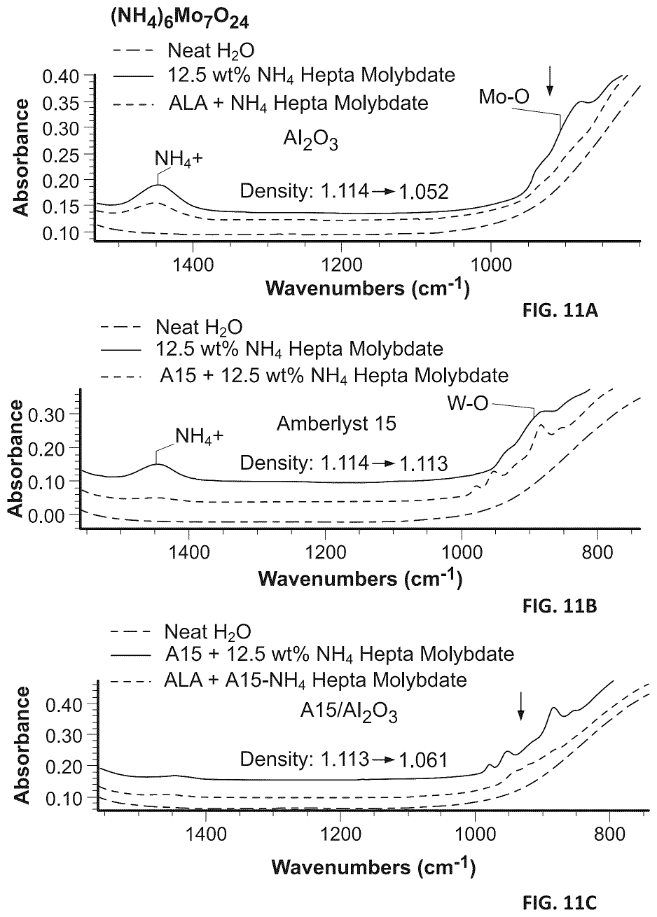

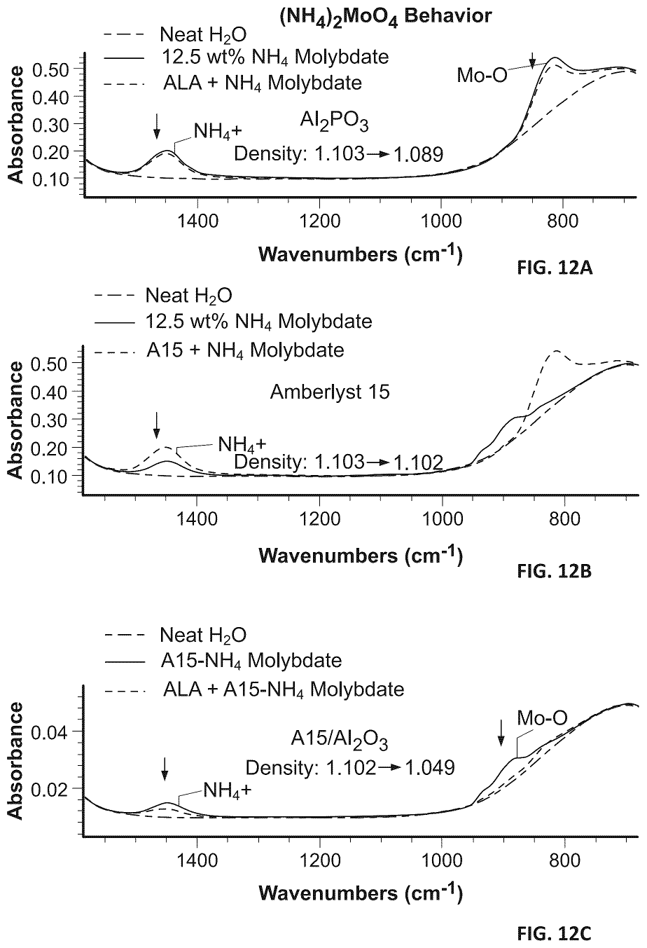

The spectroscopic and solution density behavior presented in FIGS. 9A-9C for complex tungstates such as metatungstates and FIGS. 10A-10C for simple tungstates such as Na₂WO₄ was mirrored by similar behavior of corresponding molybdate salts. The relevant spectroscopic and density data are shown in FIGS. 11A-11C and FIGS. 12A-12C. As referred to earlier and as will be shown later, despite the excellent molybdate/Al₂O₃ loadings displayed in FIGS. 11A and 11C and FIGS. 12A and 12C, no catalytic activity was observed.

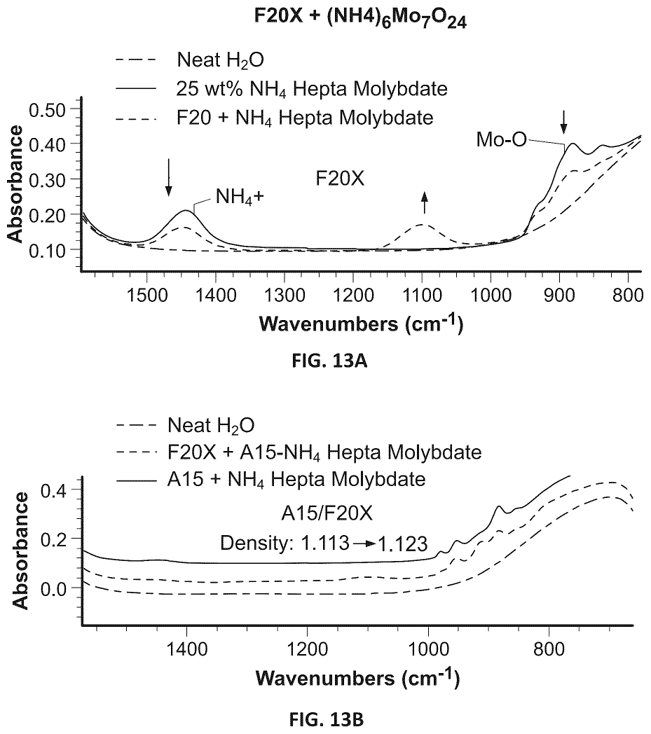

The behavior of bentonite F20X as support with tungstates and molybdates, both “as is” and acidified versions was spectroscopically more complex. One consistent finding was that a new peak at about 1100 cm⁻¹ appeared in solution slurries. This peak was tentatively identified as SO₄²⁻ ion and is hypothesized to be associated with residual sulfate on commercial bentonite F20X that is being leached off during slurrying with tungstates and molybdates. In some instances, the solution density after slurrying with tungstates or molybdates was higher than starting solution densities as shown in Table 4 indicating that there was a net movement of material into solution, wherein bentonite F20X was added at 0.5 g per ml 0.053 M solution. Representative spectra for heptamolybdate interaction with F20X are shown in FIG. 13.

TABLE 4

Density (g/cc)

| Support | Solution | Before | After |
|---|---|---|---|
| F20X | (NH₄)₆H₂W₁₂O₄₀ | 1.145 | 1.164 |
| F20X | A15-(NH₄)₆H₂W₁₂O₄₀ | 1.151 | 1.167 |

The data set of samples prepared for catalyst activity studies is shown in Table 5. All wt % loadings were calculated from decrease in solution densities after slurrying. It should be noted that the exact ratio of support mass to tungstate or molybdate solution molarity or volume is not critical as long as there is sufficient support present to allow the maximum achievable adsorption. Thus, in the case of Table 5, 0.053M metatungstate solution or its acidified version was contacted with ALA on a 2 mL/g ALA basis. This gives a loading of about 14 wt % with excess metatungstate remaining in solution.

TABLE 5

| | | | | | Experimental Details |
|---|---|---|---|---|---|
| | | Density (g/cc) | | Loading | (all mixtures were stirred at 22° C. for 30 min. before |
| Material | Support | Before | After | (wt %) | sampling for FTIR and density measurements) |
| (NH₄)₆H₂W₁₂O₄₀ | ALA | 1.152 | 1.053 | 13.6 | 6 ml of 0.053M (NH₄)₆W₁₂O₄₀ or (NH₄)₆W₁₂O₄₀ - A15 |
| (NH₄)₆H₂W₁₂O₄₀ -A15 | ALA | 1.155 | 1.044 | 14.7 | were slurried with 3 g |
| (NH₄)₆H₂W₁₂O₄₀ | Carbon | 1.152 | 1.054 | 20.5 | 6 ml of 0.053M (NH₄)₆W₁₂O₄₀ or (NH₄)₆W₁₂O₄₀ - A15 |
| (NH₄)₆H₂W₁₂O₄₀ -A15 | Carbon | 1.155 | — | >40 | were slurried with 3 g carbon |
| Na₂WO₄ | ALA | 1.125 | 1.092 | 6.7 | 6 ml of 0.38M Na₂WO₄ or Na₂WO₄-A15 were slurried |
| Na₂WO₄ -A15 | ALA | 1.118 | 1.035 | 15.3 | with 3 g of ALA |
| (NH₄)₂Mo₄ | ALA | 1.108 | 1.089 | 2.4 | 6 ml of 0.6M (NH₄)₂Mo₄ or (NH₄)₂Mo₄-A15 were |
| (NH₄)₂Mo₄ -A15 | ALA | 1.102 | 1.049 | 10.8 | slurried with 3 g of ALA or F20X |
| (NH₄)₂Mo₄ -A15 | F20X | 1.102 | 1.085 | 3.1 | |
| (NH₄)₆Mo₇O₂₄ | ALA | 1.114 | 1.052 | 11.8 | 6 ml of 0.1M (NH₄)₆Mo₇O₂₄ or (NH₄)₆Mo₇O₂₄-A15 were |
| (NH₄)₆Mo₇O₂₄ -A15 | ALA | 1.113 | 1.061 | 10.4 | slurried with 3 g of ALA or F20X |
| (NH₄)₆Mo₇O₂₄ | F20X | —¹ | —¹ | 22.8 | |
| (NH₄)₆Mo₇O₂₄ -A15 | F20X | 1.113 | 1.123 | —² | |

¹Density measurements were not collected.
²No loading could be obtained from the solution FTIR due to complex behavior in the Mo—O absorbing region.

Corresponding data in Table 6 is associated with a 1 ml/g ALA basis and this ratio is still sufficient to just reach 14 wt % loading with essentially no tungstate remaining in solution as illustrated by the decrease in solution densities to close to zero. It should also be pointed out from the Table 6 data that adsorption is essentially complete after 5 minutes of stirring slurries at 22° C.

TABLE 6

| | | | | Experimental Details |
|---|---|---|---|---|
| | | Density (g/cc) | | (all mixtures were stirred at 22° C. for 5 min. before |
| Material | Support | Before | After | sampling for FTIR and density measurements) |
| (NH₄)₆H₂W₁₂O₄₀ | ALA | 1.145 | 1.018 | 1 mL 0.053M NH₄W or |
| (NH₄)₆H₂W₁₂O₄₀-A15 | ALA | 1.140 | 1.005 | A15-NH₄W Slurried with 1 g ALA |

Acidic Alumina (ALA) vs. Basic Alumina (ALB)

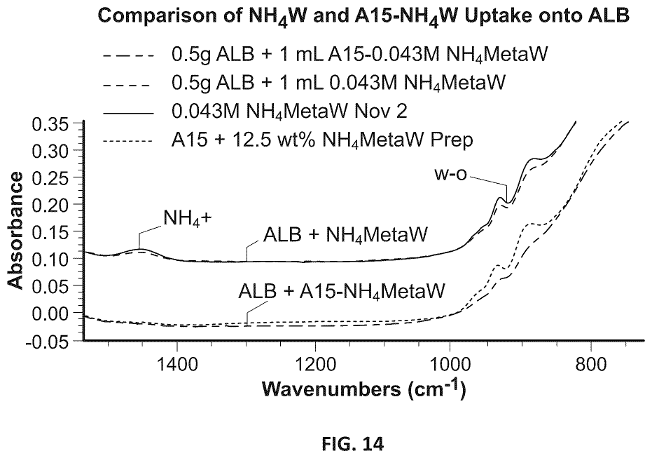

As referred to earlier, basic alumina (ALB) showed much poorer uptake of tungstate and correspondingly poorer catalytic activity than acidic alumina (ALA). Application of the acidification step to ALB showed that a correspondingly high loading to that for ALA could now be achieved. This is shown in Table 7 and FIG. 14.

TABLE 7

| | | | | | | | Experimental Details |
|---|---|---|---|---|---|---|---|
| | | | | | | | (all mixtures were stirred at 22° C. |
| | Molarity (M) | | | Density (g/cc) | | | for 5 min. before sampling for |
| Reaction | Start | Finish | Consumed | Before | After | Wt % | FTIR and density measurements) |
| ALB + (NH₄)₆H₂W₁₂O₄₀ | 0.053 | 0.0379 | 0.0151 | 1.146 | 1.102 | 8.2 | 2 g Support + 4 ml 0.053M NH₄W |
| ALA + (NH₄)₆H₂W₁₂O₄₀ | 0.053 | 0.0223 | 0.0307 | 1.146 | 1.063 | 15.2 | or A15-NH₄W Solution |
| ALB + A15 − (NH₄)₆H₂W₁₂O₄₀ | 0.053 | 0.0216 | 0.0314 | 1.141 | 1.065 | 14.6 | |
| ALA + A15 − (NH₄)₆H₂W₁₂O₄₀ | 0.053 | 0.0198 | 0.0332 | 1.141 | 1.059 | 15.4 | |

### Summary of Catalyst Preparation Findings

Metatungstate and acidified metatungstate have similar Al₂O₃ loadings. This suggests that metal oxo salts and and analogous metal oxo acids will produce similar loadings on Al₂O₃ supports.

WO₄ and MoO₄ salts have much lower Al₂O₃ loadings than their metatungstate and heptamolybdate salt analogs. This suggests that catalysts made with metal oxo salts having more than one metal atom will have higher activity than catalysts made with metal oxo salts having one metal atom.

Acidified WO₄ salts and acidified MoO₄ salts have much higher Al₂O₃ loadings than their analogous WO₄ salts and MoO₄ salts. This suggests that catalysts made with acidified metal oxo salts having one metal atom will have comparable activity to catalysts made with acidified metal oxo salts having one metal atom.

Basic alumina exhibits a poor metatungstate loading relative to acidic alumina but has comparable loading of acidified metatungstate.

Similar trends to those above described for loadings on Al₂O₃ also apply to F20X.

Both Amberlyst 15 acidifications and Al₂O₃ impregnations are essentially complete after 5 minutes at 22° C.

### Rate Acceleration Data

Table 8 contains rate acceleration data of polymer (1:1 HDPE:PP) at 400° C. pertinent to the invention. The data are divided up into paired sets associated with “as is” tungstates or molybdates and their acidified versions. A perusal of the data shows that a 3-fold rate acceleration is obtained for Al₂O₃ impregnated acidified metatungstate or tungstate over their “as is” analogs. This does not hold true for the corresponding heptamolybdate or simple molybdate salts as neither the acidified or “as is” versions show any activity. In contrast, the acidified heptamolybdate when impregnated on F20X, shows a 3-fold rate acceleration.

TABLE 8

| | | Calcination | Rate |
|---|---|---|---|
| Tungstate/Molybdate¹ | Support | Temp.² (° C.) | Acceleration |
| (NH₄)₆H₂W₁₂O₄₀ | ALA | 250 | 3.3 |
| (NH₄)₆H₂W₁₂O₄₀ | ALA | 500 | 3.6 |
| (NH₄)₆H₂W₁₂O₄₀ -A15 | ALA | 250 | 8.9 |
| (NH₄)₆H₂W₁₂O₄₀ -A15 | ALA | 500 | 8.7 |
| (NH₄)₆H₂W₁₂O₄₀ | F20X | 250 | 6.6 |
| (NH₄)₆H₂W₁₂O₄₀ -A15 | F20X | 250 | 4.9 |
| Na₂WO₄ | ALA | 250 | 1.1 |
| Na₂WO₄ -A15 | ALA | 250 | 3.3 |
| (NH₄)₆Mo₇O₂₄ | F20X | 500 | 1.1 |
| (NH₄)₆Mo₇O₂₄ -A15 | F20X | 250 | 3.6 |
| (NH₄)₆Mo₇O₂₄ | ALA | 250 | 0.8 |
| (NH₄)₆Mo₇O₂₄ -A15 | ALA | 250 | 0.7 |
| (NH₄)₂Mo₄ | ALA | 250 | 0.9 |
| (NH₄)₂Mo₄ -A15 | ALA | 250 | 0.8 |
| (NH₄)₆H₂W₁₂O₄₀ | Carbon | 250 | 1.1 |
| (NH₄)₆H₂W₁₂O₄₀ -A15 | Carbon | 250 | 1.3 |
| (NH₄)₆H₂W₁₂O₄₀ | ALB | 250 | 1.6 |
| (NH₄)₆H₂W₁₂O₄₀ -A15 | ALB | 250 | 8.0 |

¹Samples were obtained from the preparations described in Table 5.
²Post analysis, the slurries were centrifuged, the solid washed 2x with 10 ml of water and then calcined at either 250° C. or 500° C. prior to testing as depolymerization catalysts.

For the sake of brevity, only certain ranges are explicitly disclosed herein. However, in addition to recited ranges, any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, within a range includes every point or individual value between its end points even though not explicitly recited. Thus, every point or individual value may serve as its own lower or upper limit combined with any other point or individual value or any other lower or upper limit, to recite a range not explicitly recited.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the processes, machines, means, methods, and/or steps described in the specification. As one of the ordinary skill in the art will readily appreciate from the disclosure of the present invention, processes, machines, means, methods, and/or steps, presently existing or later to be developed that perform substantially the same function or achieve substantially the same result as the corresponding embodiments described herein, may be utilized according to the present invention. Accordingly, the appended claims are intended to include within their scope such processes, machines, means, methods, and/or steps.

What is claimed is:

1. A method for preparing a supported catalyst, the method comprising:
a) dissolving a metal oxo salt in a solvent to form a first solution comprising cations and metal oxoanions;
b) adding a Brønsted-Lowry acid to the first solution in an amount sufficient to react with at least a portion of the metal oxoanions to form a second solution comprising a metal oxo acid; and
c) contacting a support material with the second solution, wherein the support material is reactive with the metal oxo acid to form the supported catalyst;
wherein:
the metal oxo salt is a tungstate salt and the support material is an alumina; or
the metal oxo salt is a molybdate salt and the support material is a smectite clay.

2. The method of claim 1, wherein the tungstate salt and the molybdate salt each independently comprises one a more cations selected from the group consisting of: sodium, potassium, calcium, ammonium, lead, copper(II), iron(II), manganese(II), zinc, cadmium, silver, and cobalt.

3. The method of claim 1, wherein the tungstate salt and the molybdate salt each independently comprises sodium and/or ammonium.

4. The method of claim 1, wherein the Brønsted-Lowry acid is a strong acid.

5. The method of claim 4, wherein the Brønsted-Lowry acid is in the form of an ion exchange resin, having an exchange capacity, either stated in commercial specifications or measured in a laboratory, wherein:
a) a mass of ion exchange resin in water would then be calculated to produce an aqueous solution having a proton concentration in mmols H+/g (“PC”), corresponding to the mass of mass of ion exchange resin;
b) a mass of tungstate salt or molybdate salt to be acidified would have a number of moles of metal or ammonium cationic groups (CG+) to be exchanged in mmols CG+/g (“CG”), corresponding to the mass of tungstate salt or molybdate salt, respectively; and
c) the second solution contains sufficient ion exchange resin such that the ratio PC/CG is in the range of from 0.5-5.

6. The method of claim 4, wherein the Brønsted-Lowry acid comprises a polystyrene-divinylbenzene sulfonated resin, a gel type sulfonated polystyrene-divinylbenzene resin, a macroporous sulfonated polystyrene-divinylbenzene resin, a phenolic-based sulfonic acid resin, a crosslinked polystyrene sulfonated resin with high acid capacity, a surface sulfonated crosslinked polystyrene resin, an acrylic matrix sulfonated resin, a high crosslink density polystyrene-divinylbenzene sulfonated resin, or a combination thereof.

7. The method of claim 1, wherein:
a) the alumina support comprises gamma alumina, eta alumina, theta alumina, alpha alumina, or a combination thereof; and/or
b) the smectite clay support comprises bentonite, montmorillonite, beidellite, nontronite, saponite, hectorite, sauconite, swinefordite, or a combination thereof.

8. The method of claim 1, wherein the alumina support is acidified.

9. The method of claim 1, wherein the first solution comprises the metal oxo salt in an amount in the range of from 0.01 M to 0.20 M.

10. The method of claim 1, wherein the steps a), b), and/or c) are performed:
a) at a temperature in the range of from 1° C. to 99° C., from 10° C. to 50° C., or from 20° C. to 25° C.; or
b) at a pressure in the range of from 1 bar-a to 7 bar-g; or
c) a combination thereof.

11. The method of claim 1, wherein the solvent is water.

12. A supported catalyst for depolymerizing polymers, the supported catalyst comprising:
a) a tungstic acid supported on an alumina support; or
b) a molybdic acid supported on a smectite clay support; or
c) a combination thereof.

13. The supported catalyst of claim 12, wherein the tungstic acid and/or the molybdic acid are at least 50% protonated, at least 60% protonated, at least 70% protonated, at least 80% protonated, at least 90% protonated, or fully protonated.

14. The supported catalyst of claim 12, wherein the tungstic acid and/or the molybdic acid are isopoly acid comprising 1 to 20 metal atoms.

15. The supported catalyst of claim 12, wherein:
a) the tungstic acid is fully protonated, and the supported catalyst demonstrates a first pyrolysis rate (PR1);
b) a comparative catalyst comprising a tungstate salt corresponding to and in place of the tungstic acid demonstrates a second pyrolysis rate (PR2); and
c) PR1/PR2 is greater than or equal to 1.5, greater than or equal to 2.0, greater than or equal to 2.5, or greater than or equal to 3.0.

16. The supported catalyst of claim 12, wherein:
a) the molybdic acid is fully protonated, and the supported catalyst demonstrates a first pyrolysis rate (PR1);
b) a comparative catalyst comprising a molybdate salt corresponding to and in place of the molybdic acid demonstrates a second pyrolysis rate (PR2); and
c) PR1/PR2 is greater than or equal to 1.5, greater than or equal to 2.0, greater than or equal to 2.5, or greater than or equal to 3.0.

17. The supported catalyst of claim 12, wherein:
a) the alumina support comprises gamma alumina, eta alumina, theta alumina, alpha alumina, or a combination thereof; and
b) the smectite clay support comprises bentonite, montmorillonite, beidellite, nontronite, saponite, hectorite, sauconite, swinefordite, or a combination thereof.

18. The supported catalyst of claim 12, wherein:
a) the tungstic acid is supported on an alumina support in an amount greater than 8 wt %, or in the range of from 9 wt % to 20 wt %; and/or
b) a molybdic acid salt supported on a smectite clay support in an amount greater than 8 wt %, or in the range of from 9 wt % to 20 wt %;
wherein weight percent is based on the total weight of the supported catalyst.

19. A process for depolymerizing polymers, the process comprising:
a) adding a polyolefin-based feed stream and the supported catalyst of claim 12 to a pyrolysis reaction zone to form a mixture; and
b) reacting the mixture under depolymerization conditions in the absence of oxygen to form a first vapor stream and first liquid stream comprising char; and
c) adding the first vapor stream to a condensation zone wherein heat is removed to form a second vapor stream and a second liquid stream comprising one or more olefin monomers.

20. The process of claim 19, wherein:
a) the depolymerization conditions comprise a temperature in the range of from 250° C. to 600° C.; or
b) the conditions in the condensing zone comprise a temperature in the range of from 20° C. to 100° C.; or
c) a combination thereof.
